(19) United States
(12) Patent Application Publication
(10) Pub. No.: US 2020/0039858 A1
(43) Pub. Date: Feb. 6, 2020

(54) HORIZONTAL ANAEROBIC DIGESTOR WITH SEDIMENT SEPARATOR FOR THE ORGANIC FRACTION OF MUNICIPAL SOLID WASTE AND RELATED PROCESS

(71) Applicant: G-META CONSULTORIA PARTICIPAÇÕES E SERVIÇOS LTDA, Rio de Janeiro (BR)

(72) Inventors: GIOVANNI COASSIN, Rio de Janeiro (BR); Luciano Pesce, Casella Genova (IT)

(21) Appl. No.: 16/529,834

(22) Filed: Aug. 2, 2019

(30) Foreign Application Priority Data
Aug. 3, 2018 (BR) 102018015929-1

(51) Int. Cl. C02F 11/04; C12M 1/107; C12M 1/06; B01F 7/00

(52) U.S. Cl. CPC C02F 11/04; C12M 21/04; C02F 2203/006; B01F 7/00391; C12M 27/06

(57) ABSTRACT

The solution proposed by the present invention consists of the different shape of the bottom of a horizontal digester (1) having at the terminal part of its length (the end side), at the opposite end from the feed inlet side, a sediment separator (9) which is in the shape of a conic pyramid (91), a linear “V” (92), or a mixed form (96), which is emptied periodically, allowing the removal of the sediments deposited on the bottom. In the conic pyramidal sediment separator (91) the emptying occurs by gravity through a controlled valve (93), while in the V-shaped linear sediment separator (92) or the mixed-form sediment separator (96) the emptying is by means of a helical impeller (94) at the bottom. The sediment separator allows the use of an organic fraction of municipal solid waste coming from a less sophisticated selection process with a greater presence of residual inorganic waste.

### FIELD OF THE INVENTION

The present invention relates to an efficient processing system of the Organic Fraction of Municipal Solid Waste (OFMSW) for biodigestion in order to obtain biogas for subsequent use for generation of electrical energy, heating, or refining into bio-methane.

This avoids landfill disposal of the organic component which is the most polluting part of the waste, thereby prolonging the useful life of landfills and reducing contamination of the soil, water and air.

### BACKGROUND OF THE INVENTION

The process of anaerobic biodigestion has been widely known, studied and applied since ancient times, mainly for the treatment of residues of civil housing (sewage), agriculture, livestock and pig farming.

Most applications relate to use of biodigestion in the agricultural and agricultural sector, for example swine and cattle waste and specific crops.

For each type of waste there is a potential for biogas production, dependent on the percentage of volatile solids contained in that waste.

Relatively recent is the processing of organic waste (OFMSW), mainly in Europe.

The literature presents several studies and industrial applications that show good energy use despite some criticisms. Thus, anaerobic biodigestion is now considered a sustainable way to dispose of waste.

Several systems have been developed and even patented with respect to their process technologies, especially in relation to the dry, semi-dry, wet or particular state in which the organic wastes are processed in the digesters.

The major anaerobic digesters today used for OFMSW (which generally contain between 20% and 40% of volatile solids) are of the DRANCO, KOMPOGAS and VALORGA type and are thermophilic (i.e. they work at temperatures around 55° C.).

DRANCO and VALORGA are vertical reactors while KOMPOGAS is horizontal. All are of the “Plug and Flow” type, i.e. loading of waste at the inlet can occur only if extraction of treated waste at the outlet is successful.

In the Dranco process, the fresh waste is introduced into the reactor through its upper part and the digestate is extracted from the lower part. There is no specific mixing process in the reactor. Mixing occurs as the flow of the waste is forced downward. Part of the extracted matter is reintroduced at the inlet to the digester and mixed with fresh incoming residue, and the remainder of the digested material is dehydrated to produce the resulting organic compost.

The Kompogas process is similar to Dranco, but the movement of residue in digestion occurs in a horizontal cylindrical reactor, which has a stirrer.

In the Valorga process, the reactor is a vertical cylinder partially divided by a vertical wall. Its feed is close to the bottom of the reactor. The mass undergoing digestion moves slowly around this vertical wall and leaves on the opposite side. On the lower surface of the reactor is a set of nozzles that recirculate the biogas being generated, thereby creating, with the gas bubbles, a pneumatic mixing of the residue undergoing digestion.

### BRIEF DESCRIPTION OF THE STATE OF THE ART

### a) Bibliographic

BRAUN, R.; Weiland, P. & Wellinger, A. (2009) Biogas from Energy Crop Digestion, IEA Bioenergy, Task 37
FERNANDO LUCIANO MERLI DO AMARAL (2004) “Biodigestion of municipal solid waste: a current technological panorama,” Institute of Technological Research of the State of Sao Paulo
DANIELE OLMETTO (2008) “Anaerobic codigestion of the sludge from purification of the organic fraction of municipal waste,” Alma Mater Studiorum—Universita di Bologna Facolta di Ingegneria Corso di Laurea in Ingegneria per 1′ Ambiente e it Territorio—Tesi di Laurea in Ingegneria Sanitaria Ambientale LS

### b) Patents

Brazilian Patent PI0617206-7
Brazilian PATENT PI 0600734-1
European patent 2248886 A2
U.S. Pat. No. 4,274,838
U.S. Pat. No. 7,659,108 B2

### ADVANTAGES OF THE INVENTION

The main problems of the state of the art have been:

For DRANKO, the absence of mixers, which does not guarantee the complete reaction of the substrate with the recirculated inoculum.

For VALORGA, nozzles that require maintenance to prevent clogging.

For KOMPOGAS, the sedimentation of the heavy components in the bottom of the digester, blocking the extraction of the material at the outlet.

It should be noted that the density of the material at the inlet of the digester progressively decreases along the length of the digester due to the biological reactions that transform the volatile solids into biogas, causing the precipitation of the inert non-volatiles in the final part if the digester near the extraction pumps.

The solution proposed by the present invention consists of the different shape of the bottom of a horizontal digester (1) having at the terminal part of its length (the end wall), at the opposite end from the feed inlet side (the feed wall), a sediment separator (9) which is in the shape of a conic pyramid (91), a linear “V” (92), or a mixed form (96), which is emptied periodically, allowing the removal of the sediments deposited on the bottom. This eliminates the need for use of up to 20% of a bulking agent (shredded/ground branches from tree pruning) in the digester feed in order to maintain the density of the digestate as it travels along the horizontal digester, thereby reducing sedimentation of the heavy inorganic fraction.

While the bulking agent reduces sedimentation in this way, it unnecessarily increases the volume of the discharged material because it is indigestible itself. In addition, use of a bulking agent increases costs of operation and logistics resulting from the fact that the waste is usually found in urban areas, while the bulking agent is more available in rural areas.

In addition, the sediment separator allows the use of OFMSW from a less sophisticated selection process with a higher presence of residual inorganic waste. Another benefit results from the greater amount of digestible organic material in the feedstock, with a relative increase in production of biogas.

### DESCRIPTION OF THE DRAWINGS

FIG. 1 shows a schematic drawing of the horizontal anaerobic digester (1) composed of:

a) an axial mixer (2) with a central rotating axle (21) and a plurality of radial angularly phased blades (22),
b) A means for heating (3),
c) A feed line (4) of the organic load (OFMSW) at the inlet to the digester,
d) An extraction duct (5) for the digested material (digested OFMSW) at the digester outlet,
e) An upper biogas accumulation chamber (6),
f) A means for recirculation (7) of a fraction of the digestate,
g) A structural body of the digester (8),
h) An end wall of the digester body (81)
l) A feed wall (82) of the digester body,
1) A bottom of the digester body (83),
m) A sediment separator (9) whose walls are of a conic pyramidal shape (91)
n) An extraction valve (93) for the sediment
o) A vibrating device (95)
p) An extraction collector (10) for the light fraction of the digestate

FIG. 2 shows an axonometric view of the digester body (8) showing the vertical walls (84), the trapezoidal shape of the bottom (85), and the sediment separator (9) having a conic pyramidal shape (91) with an outlet valve (93) on the bottom.

FIG. 3 shows an axonometric view of the terminal part of the digester showing the axle (21) and the blades (22); in the lower part is represented the sediment separator (9) in its linear V configuration (92) which embodies an inverted triangular prism with a helical impeller (94) for extraction of the sediment and a density level sensor (97)

FIGS. 4.1, 4.2 show two axonometric views of a variant of the mixed sediment separator (96) which is a combination of the pyramidal and linear V shape with a helical impeller (94) for extraction of the sediment and a vibrating device (95) to facilitate settling of the sediment.

### DETAILED DESCRIPTION OF THE INVENTION

The present invention relates to a horizontal anaerobic digester whose design solves the problems of the processing of the Organic Fraction of Municipal Solid Waste (OFMSW) without adding to the load at the inlet of the digester up to 20% of bulking material (shredded tree pruning waste) needed in order to maintain high density of the digestate, thereby reducing sedimentation, especially in the final part near the extraction pump.

The innovative idea behind the invention is not to counteract the natural phenomenon of precipitation of inert materials resulting from the transformation of the Volatile Solids into Biogas with the consequent progressive decrease in the density of the organic material (along the length of the bacterial processing within the digester), but rather to modify the equipment to effectively deal with said phenomenon.

This is achieved by placing in the end part of the bottom of the digester body (83) a sediment separator (9) which collects and evacuates the inert materials that precipitate.

It should be remembered that organic matter is composed approximately of water and solids (80% volatiles and 20% inerts): for example in a case having 50% water in OFMSW the volatile solids are 40% of the total amount of the OFMSW and therefore the drop in density of the digestate is an indicator of the positive evolution of Biodigestion that is converting the volatile solids to biogas.

In many actual urban situations differentiated collection of wastes at the origin is not implemented and, therefore, it is common to use OFMSW originating from mechanical separation that contains a significant percentage of inorganic contaminants.

In this case it is evident that the process carried out by the digester that is the object of the present invention, which does not inhibit precipitation, allows hydraulic separation of the heavy contaminants which are collected and disposed of by the sediment separator (9).

In the same way the reduction of density allows the flotation of the light contaminants (generally plastics) in the upper part of the digester where they can be captured by means of a collector (10) at a level near to but below the level of the biogas.

The extraction duct for the digested material (5) at an intermediate height allows the collection of a material that is uncontaminated and therefore suitable for obtaining good-quality compost.

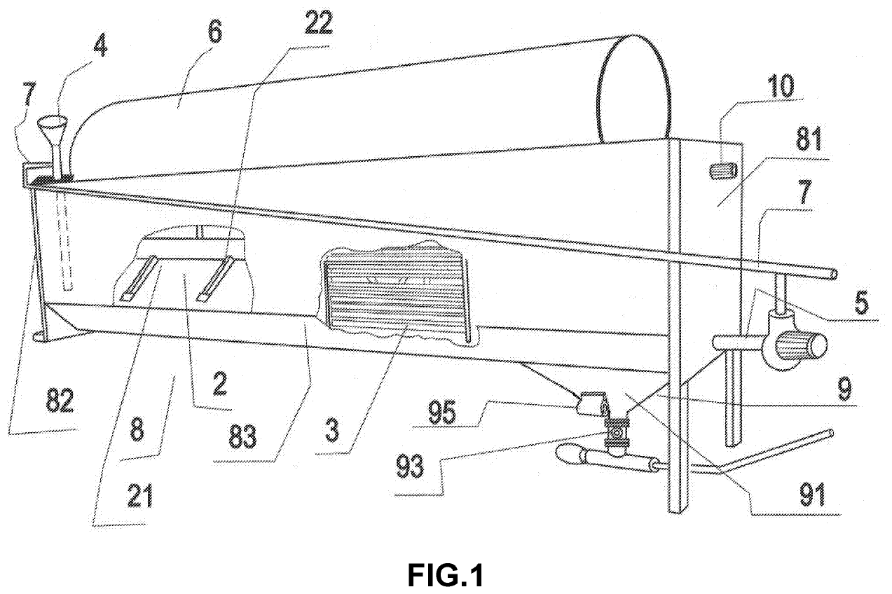

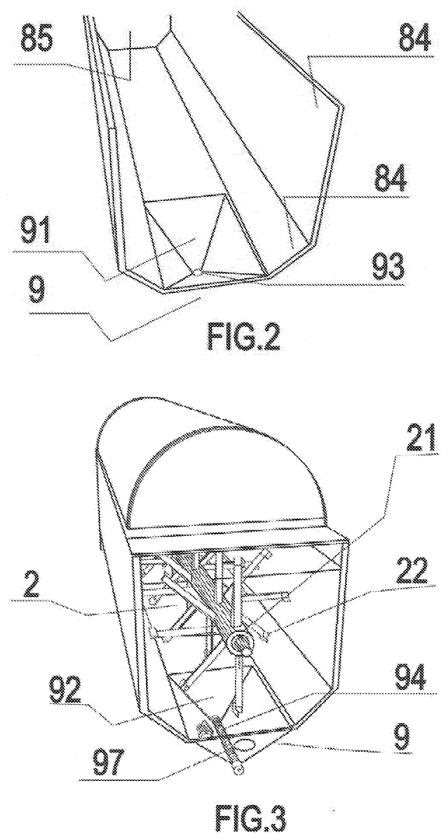

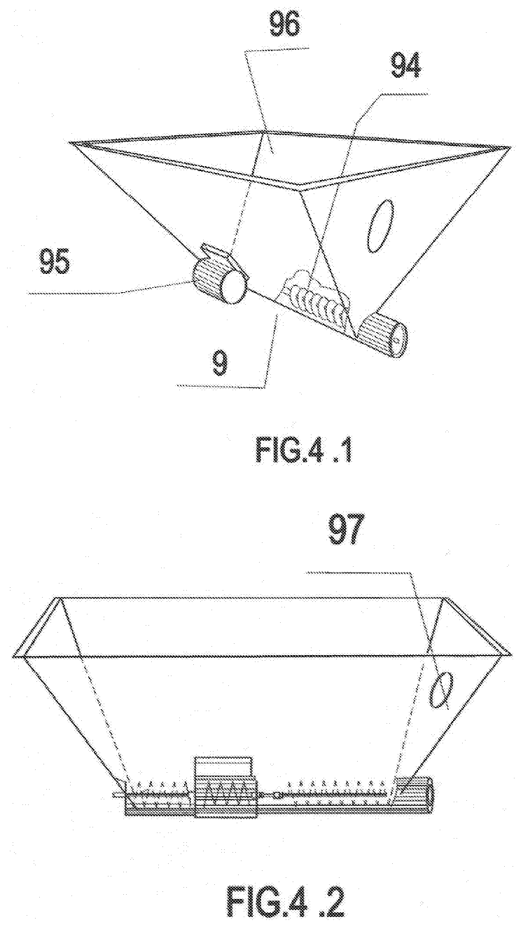

As shown in FIG. 1, OFMSW enters the horizontal anaerobic digester (1) by means of the feed duct (4), where it is mixed with a fraction of the recirculated digested material collected with the mechanism for recirculation (7) at the end of the digester where the outlet of the extraction duct (5) is located. The incoming material (OFMSW) is semi-solid and viscous and is advanced by slow rotation of an axial mixer (2) formed by a central rotating axle (21) with a plurality of blades (22) which are radially positioned therefrom and have irregular angular positioning.

U.S. Pat. No. 7,659,108 B2 discloses a central axle of this type, focusing on a flotation mechanism (gas inside the hollow axle) that solves the problem of flexing of the axle in the vertical plane but does not solve the problem of flexing in the horizontal plane.

For this reason it is safer to use a central bracket (10) to support the axle and/or use two half-axles with possibility of independent rotation in order to have better agitation in the final section of the digester where the digestate is more fluid.

The means for heating (3) placed along the vertical walls (84) of the digester body guarantee a temperature of 55° C. in the organic mass being processed, allowing the development of the thermophilic bacterial biodigestion process along the length of the horizontal digester.

The consequence of this is the transformation of volatile solids into Biogas that is collected in the upper biogas accumulation chamber (6), and the progressive decrease in density of the digestate, which is high close to the feed wall (82) and low in the vicinity of the end wall (81) where, according to the present invention, a sediment separator (9) is located in the bottom of the digester body (83).

The walls of the sediment separator (9) may have a conic pyramidal shape (91) or a linear V-shaped configuration (92) embodying an inverted triangular prism.

The inclined walls of the sediment separator (9) form an angle with the horizontal plane of the bottom (83) of the digester body between 30° and 60°.

The length of the sediment separator (in the longitudinal direction) which abuts the end wall (81) of the digester body has a value D between 15% and 33% of the length L of the digester. The removal of sediment is by gravity through extraction valve (93) at the bottom, in the case of the conic pyramidal solution (91), and with a helical impeller (94) in the bottom, in the case of the linear V (92) solution.

The present invention, for the sediment separator (9), also foresees a mixed solution (96), being a combination of the pyramidal and linear V form where the prism ends are inclined as shown in FIG. 4. In this case the helical impeller (94) has the advantage of being shorter in length.

The emptying of the sediment separator (9) is cyclic and can be controlled by a timer or can be actuated automatically by means of a density level sensor (97) which detects the filling of the sediment separator.

Additionally, the sediment separator (9) may have a vibrating device (95) on one or more of its side walls to facilitate the settling of the sedimented material to the bottom of the separator.

1. Horizontal anaerobic digester (1) comprising:
(a) an axial mixer (2) with a central rotating axle (21) and a plurality of radial angularly phased blades (22),
(b) a means for heating (3), located on the vertical walls of the digester body (84), able to maintain the temperature of the processed organic material at about 55° C.;
(c) a feed duct (4) for the organic load (OFMSW) at the inlet to the digester;
(d) a extraction duct (5) for the digestate (digested OFMSW) at the outlet of the digester;
(e) an upper chamber for biogas accumulation;
f) a means for recirculation of a fraction of the digested material, all mounted next to the digester structural body (8)
characterized by having, at the bottom of the digester body (83) and abutting the end wall (81), a sediment separator (9) with a length D (in the longitudinal direction) and with walls that are conic pyramidal (91) or linear “V” (92) or mixed (96), which, emptied periodically, allows the removal of sediment deposited at the bottom.

2. Horizontal anaerobic digester (1) according to claim 1, characterized by the fact that the numerical relationship between the length D and the total length L of the digester body (8) has a value between 0.15 and 0.33.

3. Horizontal anaerobic digester (1) according to claim 1, characterized by the fact that the sediment separator (9) has the angle between the inclined walls and the horizontal plane with a value between 30° and 60°.

4. Horizontal anaerobic digester (1) according to claim 1, characterized by the fact that in the sediment separator of the pyramidal conic shape (91) the emptying is by gravity through a controlled valve (93) and, in the sediment separators in linear V form (92) and mixed form (96) the emptying is by means of a helical impeller (94) at the bottom.

5. Horizontal anaerobic digester (1) according to claim 1, characterized by the fact that the operations for emptying the sediment separator are carried out automatically by means of a density level sensor (97) which detects the filling of the sediment separator.

6. Horizontal anaerobic digester (1) according to claim 1, characterized by the fact that the sediment separator (9) has vibratory devices (95) on one or more of its side walls to facilitate the settling of the sedimented material.

7. Horizontal anaerobic digester (1) according to claim 1, characterized by the fact that the body of the digester (8) has in the wall opposite the feed (81), at a level near to but below the level of the biogas, a collector (10) for the extraction of the light fraction consisting mainly of floating residues of plastic.

8. Horizontal anaerobic digester (1) according claim 1, characterized by the fact that the lower part of the digester body (8) has an isosceles trapezoidal section (84) with a 45° inclination.

9. Horizontal anaerobic digester (1) according to claim 1, characterized by the fact that the axle (21) has a central support so that it can be divided into 2 half-axles with independent rotation.

10. An anaerobic biodigestion process performed by treating the Organic Fraction of Municipal Solid Waste (OFMSW) in a horizontal anaerobic digester (1)
